(19) United States
(12) Patent Application Publication
(10) Pub. No.: US 2026/0047958 A1
(43) Pub. Date: Feb. 19, 2026

(54) IMPLANTING DEVICE FOR INSERTING AN IMPLANT INTO TISSUE

(71) Applicant: BOEHRINGER INGELHEIM INTERNATIONAL GMBH, Ingelheim am Rhein (DE)

(72) Inventors: Herbert Wachtel, Ingelheim am Rhein (DE); Guido Enert, Leichlingen (DE); Marty Nazzaro, Newton, MA (US)

(21) Appl. No.: 18/871,240

(22) PCT Filed: Jun. 1, 2023
(86) PCT No.: PCT/EP2023/064729

§ 371 Date: Dec. 3, 2024

(30) Foreign Application Priority Data
Jun. 3, 2022 (EP) 22177260.1

(51) Int. Cl. A61F 9/00; A61M 5/315; A61M 5/32; A61M 5/34

(52) U.S. Cl. CPC A61F 9/0017; A61M 5/3158; A61M 5/3202; A61M 5/347

(57) ABSTRACT

The present invention relates to an implanting device for inserting a plant into tissue, in particular into an eyeball.

The implanting device has a housing, a plunger received at least partly in the housing and, at an axial end of the housing, a hollow needle, and/or a connecting device for connecting a hollow needle to the housing the plunger being axially movable relative to the housing and/or the needle.

Preferably, the implanting device has an actuator for moving the plunger relative to the housing, the actuator having or forming a lever mechanism.

### BACKGROUND OF THE INVENTION

### Field of the Invention

The present invention relates to an implanting device for inserting an implant into tissue, in particular into an eyeball, according to the preamble of claim 1.

The implanting device according to the present invention is preferably adapted to insert an implant into the tissue of a patient, in particular a human or animal. In particular, the implanting device according to the present invention is adapted for intravitreal insertion or injection of the implant. In other words, the tissue is preferably the eyeball of the patient or a part thereof, in particular the vitreous body or chamber, the anterior chamber and/or the posterior chamber of the eye. The implant is in particular an intravitreal implant and/or an implant which serves to release a medicament or drug or active substance to the eye, in particular the vitreous body, over a long time span, such as several weeks or months, in particular over a time span of up to 3 years.

In general, implanting devices for inserting an implant into an eyeball are known in the art.

### Description of the Related Art

US 2013/0158561 A1 relates to an assembly for implanting ocular implants into a mammalian eye. The assembly includes a cannula having a curved distal end, an injector mechanism coupled to the cannula for linear moving one or more implant segments in the cannula towards the curved distal end and a rotator mechanism for rotating the cannula about a longitudinal axis thereof. The device may be actuated by a user pressing a release button, which drives the plunger to push implant segments along the needle by means of a compression spring.

US 2014/0031833 A1 relates to an intraocular injector including a syringe body having an open-ended cone disposed in one end thereof along with a piston disposed in the body. A transparent implant holder having a lumen therein is aligned with the open end of the cone. Injection is made through a needle with a bevel disposed on a distal end. A plunger affixed to the piston and slidable within the holder lumen and a needle lumen is provided for injecting solid intraocular implants into an eye. The plunger may be moved by pushing against a thumb pad arranged at an end of the plunger.

WO 2009/061988 A1 relates to an ocular implantation device comprising a housing having a longitudinal axis, a needle configured to receive an implant and a plunger and a rod operatively coupled together. The plunger and the rod are disposed in the housing and are collectively, translationally movable along the longitudinal axis of the housing. The rod is configured to be receivable within at least a portion of the needle to enable the rod to move an implant therethrough. An actuator is operatively engaged with the plunger such that movement of the actuator in a direction aligned with the longitudinal axis of the housing results in translational movement of the plunger the rod along the longitudinal axis of the housing in order to deliver the implant through the needle to a target site.

WO 2013/120033 A1 relates to an injector device for delivering an implant. The device includes a retracting element, a cannula needle, and a plunger. The device may comprise a latch that, when actuated by user, causes the retracting element to move the cannula needle away from the delivery site, allowing the plunger to eject the implant into the site. The device may be configured for intraocular drug delivery.

WO 2016/040605 A1 relates to an injector device for delivering a payload to an eye. The injector device comprises a housing, a cannula defining a lumen and having a proximal end received in the housing and a distal extending from the housing, a hollow needle comprising a distal end with a tip for piercing tissue, a shifter that shifts the needle from an extended position to a retracted position, and an actuator for advancing a payload from an initial position to exit from the distal end of the cannula.

### SUMMARY OF THE INVENTION

It is an object of the present invention to provide an improved implanting device which is user-friendly and/or easy, intuitive and/or safe to use and/or which is of a simple construction and/or easy and/or cost-efficient to manufacture.

The above object is achieved by an implanting device according to claim 1. Advantageous further developments are subject of the dependent claims.

The implanting device according to the present invention is preferably adapted for holding an implant and/or inserting an implant into tissue of a patient, for example a human or animal, in particular a mammal. In particular, the implanting device is adapted for inserting or injecting an implant into an eyeball, in particular the vitreous body or vitreous chamber, the anterior chamber and/or the posterior chamber of the eye.

The implanting device has a housing and a plunger, which is preferably at least partly received in the housing. The implanting device preferably has a cannula or hollow needle and/or a connecting device for connecting a cannula or hollow needle to the housing. The needle and/or connecting device is/are preferably arranged at an axial end of the housing. The plunger is axially movable relative to the housing, the needle and/or the connecting device. The hollow needle is mostly simply denoted as “needle” in the following.

According to a first aspect of the present invention, the implanting device preferably has an actuator for moving the plunger relative to the housing, the actuator having or forming a lever mechanism. This allows a simple and convenient use of the implanting device, in particular a single-handed use, wherein at the same time a simple construction of the implanting device is achieved.

According to another aspect, which may also be realized independently, the hollow needle is preferably freely rotatable around to longitudinal axis and relative to the housing. In other words, it is preferred that the needle may be positioned in any rotational position and/or that the needle is not restricted to discrete rotational positions. By this, the user may freely control or adjust the rotational angle between the needle and the housing and/or actuator, allowing for optimal control of the injection of the implant.

According to another aspect, which may also be realized independently, the implanting device preferably has a holding device for holding an implant received in the needle in position. By the holding device, it is prevented that the implant accidentally falls out of the needle before injection into the tissue or eyeball. Thus, the use of the implanting device is made easier and fail-safe.

According to another aspect, which may also be realized independently, the implanting device preferably has a cap configured to cover the needle, the cap comprising an implant lock for keeping the implant inside the needle. By this, the use of the implanting device is made easier and safer. The cap protects the needle against damage and protects a user from the needle. Further, sterility of the needle is maintained by the cap. By integrating the implant lock into the cap, it is on the one hand prevented that the implant accidentally falls out of the needle before it has been injected/inserted into the tissue or eyeball, and, on the other hand, the number of preparation steps before inserting the implant is reduced because the cap and implant lock can be removed in one common step.

Preferably, the actuator has or forms a toggle joint. The toggle joint is in particular a specific type of lever mechanism. According to the present invention, a toggle joint has or consists of two bars or arms, which are connected by a joint arranged at the ends of the arms or bars. This is conducive to a simple and cost-efficient construction. The toggle joint allows a very precise dosage of the force used for pushing the implant out of the needle and/or a very precise control of the position of the plunger. At the same time, the implanting device and in particular the actuator or actuating mechanism can be manufactured very easily and, hence, cost-effectively.

The actuator preferably has an arm forming a second class lever. The second class lever is in particular a specific type of lever mechanism. In a second class lever, the resistance or load is between the effort and the fulcrum. By the second class lever, the force needed for activating the actuator is very low and the position of the plunger can be controlled very precisely. At the same time, the actuator can be manufactured very easily and, hence, cost-effectively.

It is preferred that the actuator has an arm with a first section and a second section. Herein, the first section preferably forms a lever or bar of a toggle joint and the second section forms an actuating section. In particular, the arm having a first section and a second section is at the same time forming the second class lever. This is conducive to a simple and effortless handling or use of the implanting device and at the same time allows a simple and cost-efficient construction.

Preferably, the actuator has an arm which is configured and/or arranged to directly act on and/or move the plunger. By the arm acting on the plunger, the plunger preferably moves when the actuator is actuated. The movement of the plunger is in particular in an axial direction and/or towards the needle and/or connecting device. The arm which is configured/arranged to directly act on the plunger preferably forms one arm of the toggle joint and/or is a different arm than the arm forming the one sided lever and/or having a first and a second section.

The actuator is preferably designed as a one-piece component. In particular, the actuator has at least two arms which are preferably formed in one piece. The two arms are preferably connected by a film hinge and/or solid joint. This reduces the number of parts to be produced and also reduces the number of steps needed for assembling the implanting device.

The actuator is preferably configured to snap in with the plunger when the plunger reaches or has reached an end position. The snapping in preferably produces a tactile, visible and/or audible signal. Particularly, the snapping in provides a tactile, visible and/or audible feedback to the user of the implanting device, which in particular signals that the end position of the plunger is reached and/or the implant has been inserted into the tissue or eyeball.

The plunger is preferably latched in an initial position. Alternatively or additionally, the implanting device is configured in such a way that a force threshold has to be exceeded for moving the plunger out of the initial position and/or towards the needle and/or connecting device. In particular, the latching of the plunger causes the force threshold which is to be exceeded for moving the plunger. By this, an accidental actuation or movement of the plunger is prevented. Further, it has turned out that the latching or force threshold improves the ease of use of the implanting device because it is perceived as pleasant by users when the plunger is not directly moved upon actuation but first a force threshold has to be exceeded.

The implanting device preferably comprises a grip that is rotatably connected or connectable to the housing and connected or connectable to the needle in a rotationally fixed manner, in particular so that the needle may be rotated relative to the housing by a rotation of the grip. This allows adjusting/positioning of the needle in a desired rotational position relative to the housing, which in turn makes the implanting device pleasant to use and allows a precise control of the insertion of the implant into tissue.

The plunger and/or the housing may be arranged and/or configured in such a way that the movement of the plunger relative to the housing is impeded by friction. It has turned out that this improves the ease of use and, in particular, the user of the implanting device receives a better feedback and/or the feeling of a better control of the movement of the plunger. In particular, accidental movements of the plunger and/or accidental delivery of the implant can be prevented or the risk thereof can be reduced.

Preferably, the implanting device comprises an air passage between the plunger and the housing. The air passage in particular allows air to leave the implanting device on a side opposite the needle, implant and/or connecting device. By this, the accidental injection of air into the tissue or eyeball is prevented.

Preferably, the implanting device has at least one securing device, preferably at least or exactly two securing devices. The securing device(s) is/are in particular arranged at an (axial) end of the implanting device and/or opposite the needle and/or the connecting device. Particularly preferably, the implanting device has a first securing device for preventing an axial movement of the plunger towards the needle and/or the connecting device. Alternatively or additionally, the implanting device has a second securing device for preventing an axial movement of the plunger away from the needle and/or the connecting device. By this, accidental movements and/or actuations of the plunger can be prevented or blocked. This improves the ease of use and safety.

The implanting device preferably has a blocking device for blocking an actuation of the implanting device and/or actuator, in particular a pressing down of the actuator and/or the first arm thereof, preferably wherein the blocking device is additionally configured for preventing a movement of plunger towards the needle and/or implant. This is conducive to the security and prevents an accidental actuation. Further, assembly of the implanting device is preferably facilitated.

The housing may be at least partly translucent or transparent, in particular so that the plunger or a part thereof is visible through the housing. This enables the user of the implanting device to visually control the movement of the plunger and/or provides a visual feedback to the user. This allows a better and more precise control of the actuation and/or inserting process.

As an alternative or in addition, the implanting device or housing may have an indicator for indicating the status and/or position of the holding device and/or implant, the indicator preferably being visible through an opening of the implanting device and/or housing and/or connecting device. This enables the user of the implanting device to visually control the implanting process and/or provides a visual feedback to the user. This allows a better and more precise control of the actuation and/or inserting process.

The implanting device preferably comprises a pusher for pushing the implant out of the needle. In particular, the plunger comprises the pusher or the pusher is arranged at or connected to the plunger. The pusher is preferably in the form of a wire and/or made of metal, in particular steel.

The holding device is preferably designed as an elastic element, in particular a spring.

Preferably, the implanting device comprises or is adapted to receive the implant. Particularly preferably, the implant is received or receivable in the hollow needle.

The above-mentioned aspects and features of the present invention and the aspects and features of the present invention that will become apparent from the claims and the following description can in principle be implemented independently from one another, but also in any combination or order.

Further aspects, advantages, features and properties of the present invention will become apparent from the claims and the following description of preferred embodiments with reference to the drawings, in which:

### BRIEF DESCRIPTION OF THE DRAWINGS

FIG. 1 shows a perspective view of a first embodiment of an implanting device according to the present invention;

FIG. 2 shows the implanting device in an exploded view;

FIG. 3 shows a schematic depiction of a lever mechanism of the implanting device;

FIG. 4 shows a section of the implanting device in an initial position;

FIG. 5 shows a section of the implanting device in an intermediate position;

FIG. 6 shows a section of the implanting device in an end position;

FIG. 7 shows a section of an end of the implanting device comprising a cap;

FIG. 8 shows a section of a part of the implanting device having a holding device;

FIG. 9 shows a section of a part of the implanting device having a holding device according to a further embodiment;

FIG. 10 shows a perspective view of the holding device according to FIG. 9;

FIG. 11 shows a part of the implanting device with a blocking device in a perspective view;

FIG. 12 shows a section of the implanting device with the blocking device;

FIG. 13 shows a second embodiment of an implanting device according to the present invention in a perspective view;

FIG. 14 shows a side view of the implanting device according to the second embodiment;

FIG. 15 shows a first section through the implanting device according to the second embodiment; and

FIG. 16 shows a second section implanting device according to the second embodiment.

### DETAILED DESCRIPTION OF THE INVENTION

In the figures, the same reference signs are used for the same or similar parts and components, resulting in corresponding or comparable properties, features and advantages, even if these properties, features and advantages are not repeatedly described.

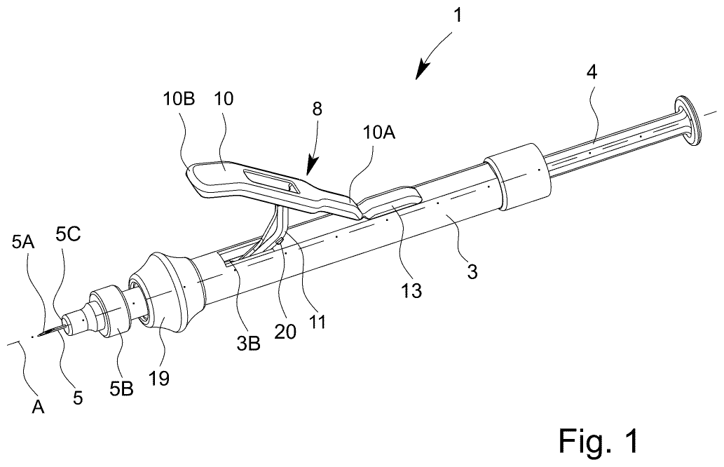

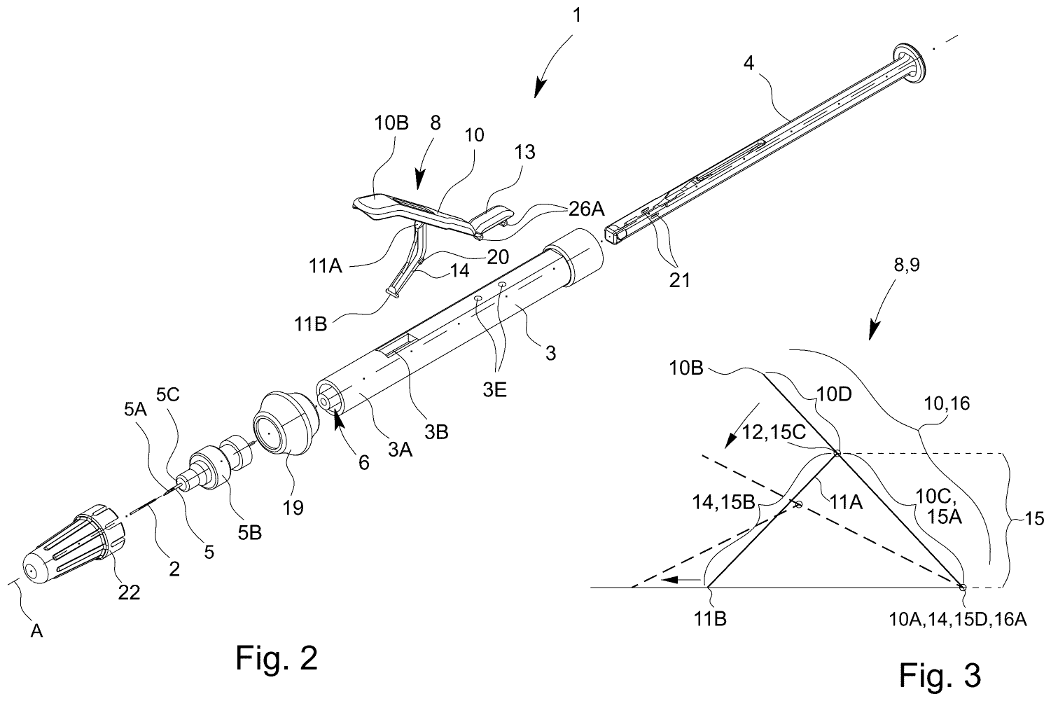

FIG. 1 shows a first embodiment of an implanting device 1 according to the present invention. In FIG. 2, the implanting device 1 according to the first embodiment is shown in an exploded view.

The implanting device 1 is configured for inserting an implant 2 into tissue of a patient (not shown). The patient is in particular a human or animal, in particular a mammal. The tissue is preferably an eyeball of the patient, in particular the vitreous body or chamber, the anterior chamber or the posterior chamber of the eye.

In other words, the present invention relates to an implanting device 1 for intravitreal injection or insertion of an implant 2.

The implant 2 is preferably an intravitreal implant. Preferably, the implant 2 comprises a medicament or drug or active substance and is in particular configured to emit the medicament or drug or active substance to the eye after insertion. In particular, the implant 2 is adapted to emit the medicament or drug or active substance over a time span of several weeks or months, in particular over a time span of up to 3 years.

The implant 2 is preferably shaped as a tube or capsule and/or comprises an impermeable or semipermeable membrane. The medicament or drug or active substance is preferably received or arranged within the membrane or capsule or tube.

Preferably, the implant 2 has a length of 10 mm or less, preferably 5 mm or less, particularly preferably 2 mm or less, and/or a width or diameter of 1 mm or less, preferably 0.5 mm or less, particularly preferably 0.3 mm or less.

The implanting device 1 has a housing 3 and a plunger 4. The plunger 4 is received at least partly in the housing 3.

The housing 3 preferably has and/or encloses an interior 3C. The plunger 4 is in particular received or receivable in the interior 3C at least partly. The interior 3C is preferably at least essentially cylindrical. The interior 3C is preferably defined or delimited by a wall 3D, in particular an inner wall, of the housing 3.

The housing 3 is preferably at least partly translucent and/or transparent. In particular, the plunger 4 or at least a part thereof is visible through the housing 3.

The implanting device 1 has a longitudinal axis A. Terms referring to an axis, such as “axially”, “axial end”, “radially” and the like refer to the longitudinal axis A in the following, if not denoted otherwise.

The implanting device 1, housing 3 and/or plunger 4 is/are preferably elongated and/or extend(s) along the axis A. The implanting device 1 and/or housing 3 is/are preferably of at least essentially pen-like configuration.

The implanting device 1 preferably has a hollow needle 5 and/or (at least) a connecting device 6 for connecting a hollow needle 5 to the housing 3. In the following, the hollow needle 5 is mostly only shortly denoted as “needle 5”.

Preferably, the housing 3 comprises the connecting device 6 and/or the connecting device 6 forms a section of the housing 3. The connecting device 6 is in particular arranged at an axial end 3A of the housing 3.

On an axial end opposite the needle 5 and/or connecting device 6, the housing 3 and/or interior 3C is preferably open.

The needle 5 is preferably designed to receive the implant 2. The implanting device 1 and/or needle 5 may comprise or contain the implant 2. In particular, the implant 2 is received or receivable in the needle 5.

Preferably, the needle 5 is configured as a hypodermic needle. The needle 5 preferably has a bevel 5A.

The needle 5 is preferably coated, for example with a silicone coating. This preferably reduces the force needed for injecting the needle 5 into tissue.

The implanting device 1 and/or needle 5 preferably comprises a connecting device 5B, in particular when the needle 5 is not fixedly connected to the housing 3 and/or when the implanting device 1 and/or housing 3 comprises the connecting device 6. The connecting device 5B of the needle 5 is preferably complementary to the connecting device 6 of the implanting device 1 and/or housing 3.

The connecting device 6 is preferably designed as a Luer lock fitting, in particular a male Luer lock fitting. In this case, the connecting device 5B of the needle 5 is preferably designed as a complementary Luer lock fitting, in particular a female Luer lock fitting.

The needle 5 may be fixedly connected to the implanting device 1 and/or housing 3, i.e. connected to the housing 3 in such a way that it is not removable therefrom. However, it is preferred that the needle 5 is releasably connectable or connected to the implanting device 1 and/or housing 3, in particular via the connecting device 6 and/or the connecting device 5B. By this, the needle 5 may be interchanged and/or different needles may be used with the implanting device 1 and/or connected to the housing 3.

In use, the needle 5 is preferably connected to the housing 3 and/or the connecting device 6. However, it is not mandatory for the implanting device 1 to comprise the needle 5 in a delivery state. It is also possible that, in the delivery state, the implanting device 1 merely comprises the connecting device 6, so that different needles 5 may be connected to the housing 3 and/or connecting device 6.

The needle 5 is preferably welded to the connecting device 5B, in particular by a laser welding process. Preferably, the needle 5 is formed in one piece with the connecting device 5B or the connecting device 5B and the needle 5 form different parts of a one-piece component. This may in particular facilitate a coating process of the needle 5.

The implanting device 1, in particular the connecting device 5B, preferably comprises a contact area 5C for contacting the tissue, in particular the eyeball, upon insertion of the implant 2. The contact area 5C is preferably flat or even and/or preferably surrounds the needle 5. Preferably, the contact area 5C has an area of 0.2 cm² or more, preferably 0.4 cm² or more, more preferably 0.6 cm² or more, in particular 0.8 cm² or more, and/or 2 cm² or less, more preferably 1.5 cm² or less, in particular 1 cm² or less.

The plunger 4 is axially movable relative to the housing 3, the connecting device 6 and/or the needle 5 and/or within the interior 3C. Thus, the plunger 4 is in particular movable along the axis A. The plunger 4 is configured to push an implant 2 received in the needle 5 out of the needle 5 and/or to insert the implant 2 into the tissue or eyeball.

In particular, the implanting device 1 and/or plunger 4 comprises a pusher 7 for pushing the implant 2 out of the needle 5. The pusher 7 is preferably arranged along the axis A and/or axially movable. The pusher 7 is at least partly arranged or arrangeable inside the housing 3. In particular, the pusher 7 is movable relative to the housing 3 and/or the needle 5.

The pusher 7 is preferably functionally coupled to the plunger 4 and in particular movable towards the needle 5 and/or implant 2 by the plunger 4. Particularly preferably, the pusher 7 is fixedly connected to the plunger 4 or forms a part thereof. The pusher is in particular arranged or fixed at an axial end of the plunger 4.

In particular, by moving the plunger 4 towards the housing 3, the pusher 7 is inserted into the needle 5 and/or advanced within the needle 5. Further, the pusher 7 acts on the implant 2 and, by movement towards the axial end, pushes the implant 2 out of the needle 5 and/or inserts or injects the implant 2 into the tissue, in particular the eyeball.

The pusher 7 is preferably elongated and/or designed as a wire or rod. The pusher 7 is preferably made of metal, for example steel or stainless steel.

According to a preferred aspect, the implanting device 1 has an actuator 8 for actuating the implanting device 1. Preferably, the actuator 8 is functionally coupled to the plunger 4 and/or directly acts or is configured to directly act on the plunger 4. In particular, the actuator 8 is configured for moving the plunger 4 relative to the housing 3. Preferably, the implanting device 1 does not comprise a spring mechanism or gear wheels for transforming a movement of the actuator 8 to a movement of the plunger 4.

The implanting device 1 is in particular configured so that an ejection of the implant 2 from the needle 5 and/or an insertion of the implant 2 into the tissue or eyeball may be effected by an actuation of the actuator 8. In particular, the implanting device 1 is configured in such a way that an actuation of the actuator 8 results in a movement of the plunger 4 towards the needle 5, wherein the plunger 4 in turn moves the pusher 7 relative to the needle 5 and acts on the implant 2, thereby pushing the implant 2 out of the needle 5.

Preferably, the actuator 8 comprises or is made of plastics. The actuator 8 is preferably at least partly flexible and/or elastic. It is preferred that the actuator 8 be injection-molded, but other methods for manufacturing the actuator 8 are also conceivable, for example 3D printing.

The actuator 8 preferably has or forms a lever mechanism 9. In other words, the actuator 8 preferably has or forms at least one or preferably more levers, in particular functionally coupled levers and/or cooperating levers.

The actuator 8 preferably has at least one arm 10, 11. Particularly preferably, the actuator 8 has at least or exactly two arms 10, 11. The arms 10, 11 of the actuator 8 are in the following denoted as “first arm 10” and “second arm 11”.

The denotations “first arm” and “second arm” do not imply a sequence or order of the arms 10, 11. Further, the term “first arm” does not necessarily imply the presence of a second securing device. Likewise, the term “second arm” does not necessarily imply the presence of two arms. In particular, the actuator 8 may even comprise only one arm, in particular only the second arm 11 without having a first arm 10. The arms 10, 11 may form or realize different levers of the lever mechanism 9.

The actuator 8 preferably has a connecting section 13. The connecting section 13 is connected or connectable to the housing 3, in particular by gluing, screwing, a latching mechanism or the like.

Preferably, the arms 10, 11 each form one or more levers of the lever mechanism 9.

The first and second arms 10, 11 are preferably integrally formed and/or designed in one piece. The first arm 10 and the connecting section 13 are preferably integrally formed and/or designed in one piece. In other words, the actuator 8 is preferably a one-piece component comprising the first arm 10 and the second arm 11 and preferably also the connecting section 13.

The second arm 11 is preferably arranged between the first arm 10 and the plunger 4. Particularly preferably, the second arm 11 provides a functional coupling of the actuator 8 to the plunger 4, in particular a functional coupling between the first arm 10 and the plunger 4.

The second arm 11 is preferably configured and/or arranged to directly act on the plunger 4 so as to move the plunger 4 when the actuator 8 is actuated. In particular, the second arm 11 is configured and/or arranged to move the plunger 4 in an axial direction and/or towards the needle 5 and/or connecting device 6.

The second arm 11 may be angled or, in other words, have two different parts that are arranged at an angle to each other. In other words, the second arm 11 is preferably not realized as a straight arm.

The second arm 11 preferably reaches through the housing 3. In particular, the housing 3 comprises an opening 3B, in particular a breakthrough, through which the second arm 11 reaches or in which the second arm 11 is arranged. The opening 3B is preferably arranged laterally at the housing 3.

The second arm 11 is preferably connected to the first arm 10, in particular at a first end 11A of the second arm 11. The second arm 11 or first end 11A thereof is preferably connected to the first arm 10 by a solid joint 12. The solid joint 12 is in particular configured as a film hinge. The film hinge is in particular a thin section of the actuator 8 around which the second arm 11 may be pivoted.

The first arm 10 of the actuator 8 preferably has a fixed end 10A and a free end 10B.

The fixed end 10A is preferably connected to the housing 3 and/or the connecting section 13, in particular by a solid joint 14, the solid joint 14 preferably being configured as a film hinge. In particular, the first arm 10 is pivotable around the fixed end 10A and/or solid joint 14.

The second arm 11 is preferably connected to the first arm 10 between the fixed end 10A and the free end 10B. The solid joint 12 is preferably arranged between the fixed end 10A and the free end 10B of the first arm 10.

The first arm 10 preferably has a first section 10C and a second section 10D. The first section 10C is preferably the section between the fixed end 10A or solid joint 14 and the position at which the second arm 11 is connected to the first arm 10, in particular the solid joint 12. The second section 10D is preferably the section between the free end 10B and the position at which the second arm 11 is connected to the first arm 10, in particular the solid joint 12.

The second section 10D is in particular an actuating section of the actuator 8. In particular, the second section 10D or actuating section is thus the section of the actuator 8 which is actuatable or to be actuated by a user.

The first arm 10 is preferably slanted relative to the housing 3 and/or axis A and/or projects from the housing 3. In an embodiment not shown in the figures, the first arm 10 may be configured to be parallel to the housing 3 and/or to be positioned parallel to the housing 3, in particular in the delivery state of the implanting device 1 and/or in order to safe space.

FIG. 3 shows in a very schematic representation the lever mechanism 9, which is in particular realized or formed by the actuator 8.

According to a preferred aspect, the lever mechanism 9 is or comprises a toggle joint 15. In particular, the actuator 8 has or forms a toggle joint 15.

A “toggle joint” in the sense of the present disclosure is preferably a mechanism, in particular a mechanical linkage, comprising or consisting of two bars, namely a first bar and a second bar, which are coupled to each other by a joint. This is very schematically shown in FIG. 3, which shows the lever mechanism 9. The toggle joint 15 comprises a first bar 15A and a second bar 15B. The bars 15A, 15B are coupled to each other by a joint 15C which is in particular located at the ends of the bars 15A, 15B. The first bar 15A is fixed at a fulcrum 15D and may be pivoted around the fulcrum 15D. The second arm 15B, in turn, may be pivoted around the joint 15C. The end of the second bar 15B that is opposite the joint 15C is movable along a straight line, as schematically shown in FIG. 3. When a force acts on the first bar 15A, the first bar 15A pivots around the fulcrum 15D and the second bar 15B, in turn, pivots around the joint 15C relative to the first bar 15A. At the same time, the end of the second bar 15B that is opposite the joint 15C moves along the straight line, in particular to the left in FIG. 3. This movement is indicated by the arrows and the dashed lines in FIG. 3.

The first and second bars 15, 15B of the toggle joint 15 preferably each form a lever of the lever mechanism 9.

In the actuator 8, it is preferred that the first section 10C of the first arm 10 realizes or represents the first bar 15A of the toggle joint 15, the second arm 11 realizes or represents the first bar 15B of the toggle joint 15, the solid joint 14 realizes or represents the fulcrum 15D of the toggle joint 15 and the solid joint 12 realizes or represents the joint 15C of the toggle joint 15.

In other words, preferably the first section 10C of the first arm 10 forms the first bar 15A of a toggle joint 15 and the second arm 11 forms the second bar 15B of a toggle joint 15.

According to another preferred aspect, the lever mechanism 9 is or comprises a second class lever 16. In particular, the actuator 8 has or forms a second class lever 16.

A “second class lever” in the sense of the present disclosure is in particular a lever where the resistance or load is located between the effort and the fulcrum. A second class lever 16 with a fulcrum 16A is in particular shown in FIG. 3. The fulcrum 16A is located at a fixed end of the second class lever. In particular, the fulcrum 16A is formed by and/or identical to the fulcrum 15D of the toggle joint 15. The resistance or load is in particular formed by the second bar 15B of the toggle joint 15.

The second class lever 16 preferably forms a lever of the lever mechanism 9.

The first arm 10 of the actuator 8 preferably realizes or forms the second class lever 16. Thus, preferably, the force of a user actuating the actuator 8 in the second section 10D represents the effort and the resistance of the second arm 11 acting on the first arm represents the resistance or load of the second class lever 16.

In the first embodiment shown in FIGS. 1 to 6, the lever mechanism 9 comprises both a toggle joint 15 and a second class lever 16. In particular, the actuator 8 comprises or forms both a toggle joint 15 and a second class lever 16. Herein, the first arm 10 preferably forms the second class lever 16, with the first section 10A at the same time forming the first bar 15A of the toggle joint 15, and the second arm 11 forms the second bar 15B of the toggle joint 15. Further, the solid hinge 12 preferably forms the joint 15C of the toggle joint 15 and the solid hinge 14 preferably forms the fulcrum 15D, 16A of the toggle joint 15 and/or second class lever 16.

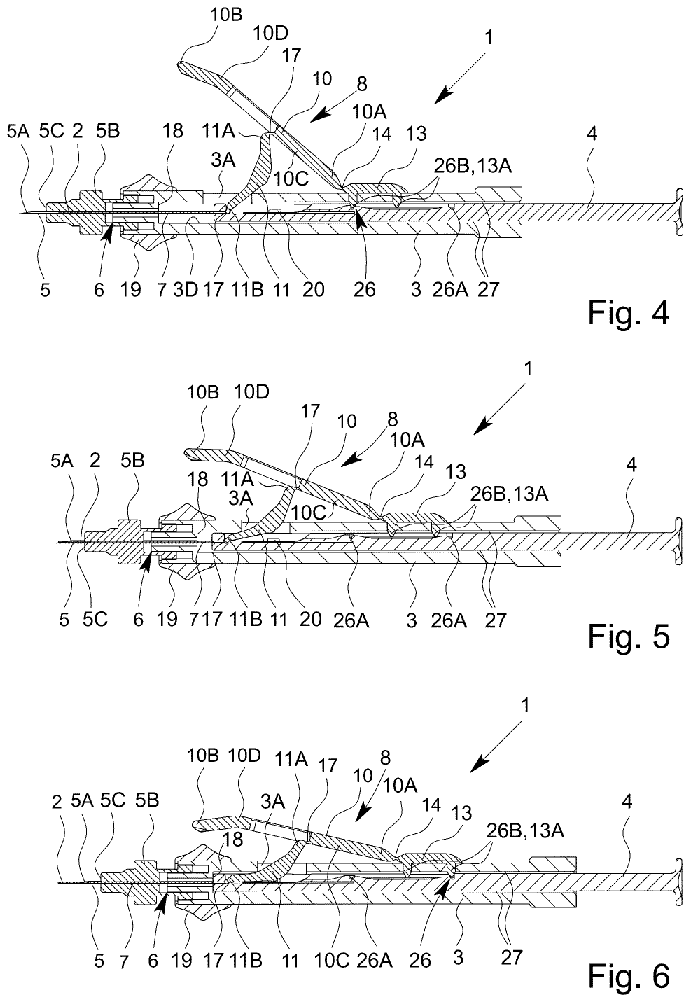

In the following, an actuation and/or motion sequence of the implanting device 1 according to the first embodiment will be described in particular in connection with FIGS. 4, 5 and 6. In FIG. 4, the implanting device 1 is shown in an unactuated, initial position. FIG. 5 shows the implanting device 1 during actuation and/or in an intermediate position. Finally, FIG. 6 shows the implanting device 1 in an end position and/or after actuation and/or ejection of the implant 2.

In the initial position, the actuator 8, in particular its first arm 10, preferably projects from the housing 3 and/or is slanted relative to the housing 3. The second end 11B of the second arm 11 preferably contacts the plunger 4. In particular, the second end 11B rests against a stop 17 of the plunger 4.

The stop 17 is preferably formed by a section of the plunger 4 extending transversally, in particular perpendicularly, to the longitudinal axis A, in particular an undercut.

In FIG. 5, the actuator 8 and/or implanting device 1 is in an intermediate position, in which the actuator 8 has been (partially) actuated, in particular by pressing on the first arm 10, particularly the second section 13D thereof. By the actuation, the first arm 10 has been moved to or pressed down towards the housing 3. By the solid joint 12 and/or the second arm 11, the force exerted on the first arm 10 upon actuation is transferred to the plunger 4 by the second arm 11 resting against the stop 17, in particular resulting in a movement of the plunger 4 towards the needle 5. By the movement of the plunger 4, the pusher 7 is moved as well and pushes against the implant 2 received in the needle 5.

In FIG. 6, the actuator 8 has been completely actuated and/or pressed down. Accordingly, the plunger 4 has been further moved, in particular into an end position. In the end position, the plunger 4 preferably rests against a stop 18 of the housing 3. Further, in the end position, the implant 2 has preferably been pushed out of the needle 5 by the pusher 7.

The actuator 8 preferably has a coupling device 20 for coupling the actuator 8 to the plunger 4. Preferably, the plunger 4 has a coupling device 21 that corresponds or is complementary to the coupling device 20 of the actuator 8.

The coupling devices 20, 21 of the actuator 8 and the plunger 4 preferably form different parts of a coupling mechanism for coupling the actuator 8 to the plunger 4.

The coupling mechanism and/or the coupling devices 20, 21 is/are preferably configured for securing or locking the actuator 8 and/or plunger 4 in the end position and/or after insertion of the implant 2, in particular by latching.

Preferably, the coupling device 20 of the actuator 8 is arranged at the second arm 11 and/or the second arm 11 comprises the coupling device 20. In particular, the coupling device 20 is arranged laterally at the second arm 11.

Particularly preferably, the coupling devices 20, 21 of the actuator 8 and the plunger 4 are configured as or form a latching mechanism. In particular, the coupling device 20 of the actuator 8 has or is formed by one or more latch cams and the coupling device 21 of the plunger 4 has or is formed by one or more corresponding or complementary notches or vice versa.

The actuator 8 is preferably configured to snap in with the plunger 4 when the plunger 4 reaches or has reached the end position. The snapping in preferably produces a tactile, visible and/or audible signal, in particular a click or the like.

Particularly preferably, the snapping in and/or the tactile, visible and/or audible signal is produced by the coupling of the actuator 8 to the plunger 4 by the coupling mechanism, in particular the latching of the coupling device 20 or latch cams into the coupling device 21 or notches.

Particularly, for latching of the coupling device 20 or latch cams into the coupling device 21 or notches, a force threshold has to be exceeded first. This force threshold in particular forms a tactile signal. Further, when the force threshold is exceeded, the coupling device 20 preferably snaps into the coupling device 21 with a quick movement. Accordingly, the first arm 10 preferably performs a quick movement when the coupling device 20 latches with the coupling device 21. This quick movement of the first arm 10 is preferably a tactile and/or visible signal.

In the following, further aspects of the implanting device 1, in particular the first embodiment thereof, will be explained in more detail, again in particular referring to FIGS. 1 and 2.

According to a preferred aspect, which may also be realized independently, the needle 5 is freely rotatable, in particular around the longitudinal axis A and/or relative to the housing 3 and/or the actuator 8. In particular, the rotational angle or position between the needle 5, particularly the bevel 5A thereof, and the actuator 8 and/or the housing 3 is adjustable, in particular freely adjustable. This may increase the ease of use of the implanting device 1 for the user and/or aid in a precise and/or comfortable insertion of the implant 2 into the tissue or eyeball.

In particular, the connecting device 6 may be, particularly freely, rotatable around the longitudinal axis A and/or the housing 3.

“Freely rotatable” in the sense of the present disclosure in particular means that the freely rotatable part, in particular the needle 5 and/or the connecting device 6, is continuously rotatable around the axis A and/or may be positioned in any desired rotational position or angle. This is in particular in contrast to a stepwise rotation, in which the rotatable part may only be positioned in defined and/or discrete rotational positions or angles.

The implanting device 1 preferably comprises a grip 19. The grip 19 is preferably arranged at an axial end of the housing 3, in particular the axial end 3A comprising the connecting device 6 and/or needle 5.

The grip 19 preferably has an annular shape. Preferably, the grip 19 is (freely) rotatable relative to the housing 3 and/or the actuator 8 and/or around the axis A. It is preferred that the grip 19 is rotationally coupled to the needle 5 and/or one or both of the connecting devices 5B, 6, in particular so that the grip 19 is rotationally fixed relative to the needle 5 and/or connecting device(s) 5B, 6 and/or that the needle 5 rotates with the grip 19. In particular, this facilitates a rotation of the needle 5 and reduces the risk of damage of the needle 5 or injuries upon rotation.

The grip 19 preferably has a larger diameter and/or cross section than the housing 3 and/or surrounds the housing 3 at least partially. Hence, the grip 19 preferably serves as a device against which user may rest one or more fingers when actuating or using the implanting device 1. This allows for a comfortable use and/or actuation of the implanting device 1 and aids in a precise insertion and/or injection of the implant 2.

Preferably, the grip 19 and/or housing 3 are configured in such a way that there is friction between the grip 19 and the housing 3 when rotating the grip 19. In other words, a rotation of the grip 19 relative to the housing 3 and/or around the axis A is preferably impeded by friction. Particularly preferably, the amount of friction between the grip 19 and the housing 3 is chosen and/or adjusted such that, on the one hand, the grip 19 may be freely and easily rotated, and, on the other hand, the grip 19 securely stays in a chosen or desired rotational position as long as it is not actively rotated by a user. This is conducive to easily and precisely adjusting a desired rotational angle or position of the needle 5 and/or bevel 5A.

The implanting device 1 preferably has a cap 22 configured to cover and/or protect the needle 5. The cap 22 is preferably connectable to the connecting device 5B of the needle 5, the housing 3, the connecting device 6 and/or the grip 19.

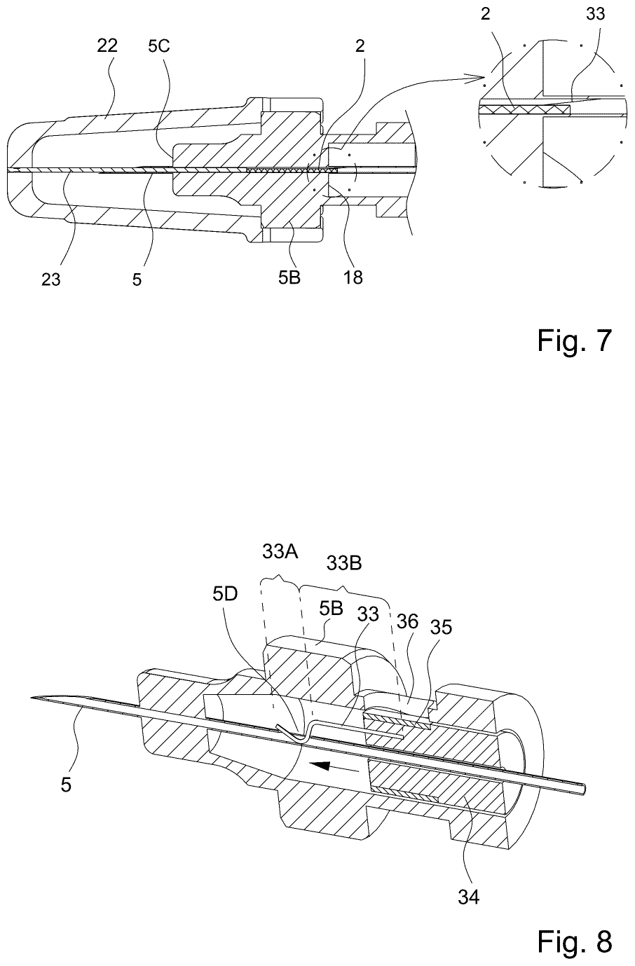

The cap 22 is in particular shown in FIG. 7.

The implanting device 1 and/or cap 22 preferably comprises an implant lock 23. The implant lock 23 is in particular configured for keeping the implant 2 inside the needle 5 and/or for preventing the implant 2 from falling out of the needle 5.

The implant lock 23 is in particular fixedly connected to the cap 22. This allows the cap 22 and the implant lock 23 to be removed from the needle 5 together and/or in a single step, thus facilitating and/or speeding up the preparation of the implanting device 1 before inserting the implant 2. The cap 22 and the implant lock 23 may be formed as a one-piece component, in particular by injection-molding the cap 22 and the implant lock 23. However, other solutions are also possible here. For example, the implant lock 23 may be glued or welded to the cap 22 or in any other way be fixedly connected to the cap 22.

The implant lock 23 is preferably insertable into the needle 5. As an alternative or in addition, however, the implant lock 23 may also be configured as a sleeve or the like for receiving the needle 5 and/or for pushing over the needle 5.

The implanting device 1 and/or needle 5 and/or connecting device 5B preferably has a holding device 33 for holding the implant 2 received in the needle 5 in position.

In FIGS. 7 to 10, several embodiments of the holding device 33 are shown. If not explicitly noted otherwise or apparent from the context, explanations with regard to one of the embodiments of FIGS. 7 to 10 also relate to the other embodiments.

An exemplary embodiment of the holding device 33 is in particular shown in the enlarged detail of FIG. 7.

The holding device 33 is preferably configured for contacting the implant 2 and/or for exerting a force or pressure on the implant 2, in particular transversally or perpendicularly to the axis A. By this, the implant 2 is preferably pressed against the inner wall of the needle 5 and/or held in position and/or prevented from falling out of the needle 5, in particular after removing the cap 22 and/or implant lock 23 and/or before injection of the implant 2 into tissue.

Preferably, the holding device 33 is arranged at least partly or completely inside the needle 5. In the example shown in FIG. 7, the holding device 33 is fixed at an inner wall of the needle 5 and in particular projects therefrom. Particularly, the holding device 33 is slanted relative to the needle 5 and/or axis A. Preferably, the holding device 33 is arranged between the implant 2 and the wall of the needle 5.

The holding device 33 is preferably designed as an elastic element, in particular a spring, particularly preferably a flat spring or cantilever spring.

The holding device 33 or spring is preferably bent, compressed and/or tensioned when holding the implant 2.

Preferably, the holding device 33 is releasable or removable from the implant 2, in particular automatically and/or before injection of the implant 2 or pushing the implant 2 out of the needle 5.

Particularly preferably, the implanting device 1 is configured for, particularly automatically, releasing the implant 2 from the holding device 33 when the plunger 4 is moved towards the needle 5 and/or implant 2.

For example, the holding device 33 may be configured and/or arranged in such a way that it is released and/or moved away from the implant 2 by the plunger 4 and/or pusher 7, at least indirectly or by one or more additional parts between the holding device 33 and the plunger 4 and/or pusher 7.

In the example shown in FIG. 7, the holding device 33 is fixed to the needle 5 at a position that is between the implant 2 and the pusher 7 and/or plunger 4. Thus, when the plunger 4 is moved towards the needle 5 and/or implant 2, the pusher 7 preferably first comes into contact with the holding device 33 or spring, thereby pushing the holding device 33 or spring outward and/or away from the implant 2 and/or axis A. By this, the holding device 3 is removed from the implant 2 and/or the implant 2 is released. Then, upon further movement of the plunger 4 towards the implant 2, the pusher 7 preferably comes into contact with the implant 2 and in particular eventually pushes the implant 2 out of the needle 5.

In the embodiment shown in FIG. 8, the implanting device 1 and/or connecting device 5B preferably comprises a holder 34. The holder 34 holds or is adapted to hold the holding device 33.

The holder 34 is preferably arranged inside the implanting device 1 and/or housing 3, in particular inside the connecting device 5B and/or interior 3C. The holding device 33 and/or the holder 34 is/are preferably movable relative to the needle 5 and/or the implant 2, which is not shown in FIG. 8. In particular, the holding device 33 and/or holder 34 is/are movable along the axis A.

Preferably, the holder 34 surrounds the needle 5 and/or is of at least essentially cylindrical shape.

The holding device 33 is preferably fixed to the holder 34 and/or movable together with the holder 34.

The holding device 33 preferably comprises a holding section 33A. The holding section 33A of the holding device 33 is preferably configured to press on the implant 2 and/or to hold the implant 2 in position in the needle 5.

In the embodiment shown in FIG. 8, the holding device 33 is preferably designed as or formed by an in particular curved or bent wire. In particular, the holding section 33A is bent or curved. Preferably, the holding section 33A has at least essentially a U shape or V shape. The holding section 33A preferably comprises two legs which are angled to each other, the angle between the legs preferably being 30° or more, more preferably 40° or more, in particular 50° or more, particularly preferably 60° or more.

Further, the holding device 33 preferably comprises a connecting section 33B for connecting the holding device 33 to the holder 34. The connecting section 33B is preferably arranged at least essentially parallel to the longitudinal axis A and/or fixed to the holder 34.

The holding section 33A and the connecting section 33B preferably lie in a common plane. The holding section 33A preferably projects from the connecting section 33B and/or is angled relative to the connecting section 33B. The angle between the holding section 33A and the connecting section 33B is preferably at least 90°, preferably at least 95° or at least 100°.

The needle 5 preferably comprises an opening 5D which is in particular arranged laterally at the needle 5. The opening 5D preferably allows the holding device 33 to reach at least partly into the needle 5 and/or to contact to the implant 2, in particular to exert a pressure on the implant 2.

The implant 2 is preferably held in position by an in particular radial pressure or force exerted on the implant 2 by the holding device 33, in particular the holding section 33A. The holding section 33A is preferably arranged or arrangeable in the opening 5D.

The implanting device 1 preferably comprises an indicator 35 for indicating the status and/or position of the holding device 33 and/or the implant 2. In particular, the indicator 35 is configured for indicating if the implant 2 is (still) secured or held in position by the holding device 33 or not. The indicator 35 is preferably an optical indicator.

For example, the indicator 35 may have a specific color or several different colors for indicating the status of the implant 2 and/or the indicator 35 may indicate the status of the implant 2 and/or holding device 33 by numbers, figures, symbols, words or the like.

In the embodiment shown in FIG. 8, the implanting device 1 or housing 3 or connecting device 5B preferably comprises at least one or more openings 36. The opening(s) 36 may be designed as a breakthrough through the connecting device 5B or housing 3 that may also have or be formed by a window or the like. Preferably, the indicator 35 and/or holder 34 is visible through the opening 36 or window.

The presence of several openings 36 in particular allows to see or read the indicator 35 in any rotational position of the needle 5 and/or holder 34.

Preferably, the holder 34 comprises the indicator 35.

The plunger 4 is preferably configured to move the holder 34 and/or holding device 33. When the implanting device 1 is actuated or the plunger 4 is moved, the holder 34 is preferably contacted by the plunger 4 or a part in operational connection therewith, so that a movement of the plunger 4 results in a movement of the holder 34 along the axis A and/or towards the end of the needle 5 (i.e., to the left in FIG. 8, as indicated by the arrow). By this movement of the holder 34, the holding device 33 is preferably also moved relative to the needle 5, so that the holding device 33, in particular the holding section 33A or a leg thereof, slides along the edge of the opening 5D and/or is lifted from the implant 2 and the holding device 33 or holding section 33A is moved out of the needle 5, so that the implant 2 is released from the holding device 33 and/or no longer secured by the holding device 33. After that, the plunger 4 or pusher 7 preferably pushes the implant 2 out of the needle 5.

The movement of the holder 34 is preferably visible for the user through the opening 36. During and/or after the movement of the holder 34, preferably different sections of the holder 34 and/or indicator 35 are/become visible through the opening 36. This is in particular an indication for the user that the implant 2 is (still) held or secured by the holding device 33 or not (anymore) held or secured by the holding device 33. For example, the indicator 35 may have to different colors and/or symbols so that different colors/symbols become visible during the movement of the plunger 4 and/or the holder 34, wherein the different colors or symbols and/or the change thereof indicate the status to the user.

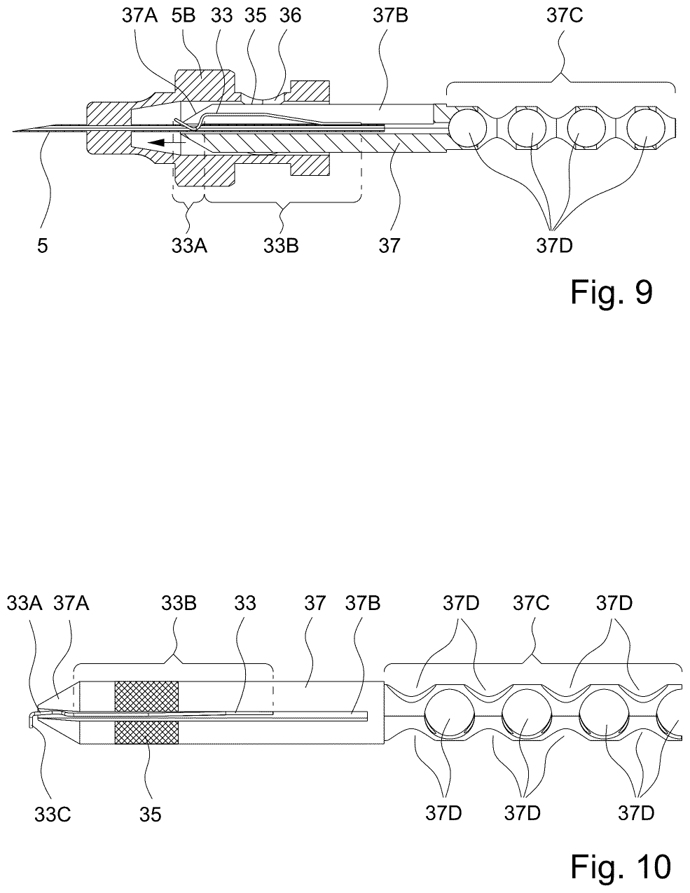

In FIGS. 9 and 10, a further embodiment of the holding device 33 is shown.

In the embodiment shown in FIGS. 9 and 10, the holding device 33 is preferably in a fixed position along the axis A and/or relative to the needle 5. In other words, the holding device 33 is preferably not (axially) movable relative to the needle 5. In particular, the holding device 33 is fixedly or immovably connected to the needle 5, at least in axial direction.

The implanting device 1 preferably comprises a releasing device 37 for releasing the holding device 33. The releasing device 37 is preferably arranged at least partly within the housing 3 or its interior 3C and/or the connecting device 5B.

The releasing device 37 is preferably movable within the housing 3, the connecting device 5B and/or interior 3C and/or relative to the needle 5 and/or the holding device 33. In particular, the releasing device 37 is movable along the axis A.

The releasing device 37 preferably comprises an indicator 35. The indicator 35 of the releasing device 37 is preferably designed as the indicator 35 of the holder 34 described before in connection with the embodiment shown in FIG. 8.

In particular, the releasing device 37 is configured for releasing the holding device 33 from the implant 2, in particular by relative movement between the holding device 33 and the releasing device 37 and/or by lifting the holding device 33 or moving the holding device 33 away from the needle 5 and/or implant 2.

The releasing device 37 preferably has a contact area 37A for contacting the holding device 33 or a section thereof, in particular the contact section 33C. The contact area 37 is preferably arranged at an axial end of the releasing device 37 and/or is slanted relative to the needle 5 and/or axis A. In particular, the contact area 37 is at least essentially in the shape of a cone or the like.

The holding device 33 preferably comprises a contact section 33C, which is in particular configured to be contacted by the contact area 37A of the releasing device 37. The contact section 33C is preferably arranged transversally, in particular perpendicularly and/or radially, to the needle 5 and/or axis A. The contact section 33C is in in particular arranged at an end of the holding device 33 and/or forms an end section of the holding device 33.

In the embodiment shown in FIGS. 9 and 10, the holding device 33 preferably also comprises the holding section 33A. The holding section 33A is preferably designed as described as the holding section 33A of the holding device 33 according to the embodiment shown in FIG. 8. The above explanation concerning the holding section 33A thus also hold for the holding section 33A of the embodiment shown in FIGS. 9 and 10.

Further, the holding device 33 preferably comprises a connecting section 33B. The holding device 33 or connecting section 33B is preferably connected to the needle 5, in particular so that the holding device 33 is in a fixed axial position relative to the needle 5. Preferably, the connecting section 33B comprises two straight sections which are preferably angled to one another or is formed thereby.

The holding section 33A is preferably arranged between the contact section 33C and the connecting section 33B.

The contact section 33C preferably runs transversally, in particular perpendicularly, to the holding section 33A and/or the connecting section 33B and/or a plane in which the holding section 33A and the connection section 33B lie.

The releasing device 37 preferably comprises a recess 37B for receiving the holding device 33, in particular the connecting section 33B and/or the holding section 33A. The recess 37B is preferably a slot or cutout in the releasing device 37. Preferably, the recess 37B extends parallel to the axis A.

The plunger 4 is preferably configured to move the releasing device 37, in particular relative to the holding device 33 and/or the needle 5. When the implanting device 1 is actuated and/or the plunger 4 is moved, the plunger 4 or a part in operational connection therewith preferably contacts the releasing device 37, so that a movement of the plunger 4 results in a movement of the releasing device 37 along the axis A and/or towards the needle 5 (i.e., to the left in FIG. 9, as indicated by the arrow). By this movement of the releasing device 37, the contact area 37A contacts the holding device 33, in particular the contact section 33C. Upon further movement of the plunger 4 and/or releasing device 37, the contact section 33C slides along the moving contact area 37A, resulting in the holding section 33A and the contact section 33C being lifted and/or moved away from the needle 5 and/or implant 2. Thus, the implant 2 is released from the holding device 33 or holding section 33A.

The releasing device 37 preferably comprises a spring section 37C. The spring section 37C is preferably integrally formed or formed in one part with the releasing device 37.

The releasing device 37 and/or the spring section 37C is/are preferably formed from a flexible and/or elastic material, in particular plastics.

The spring section 37C is in particular compressible, particularly preferably along the axis A. By this, the total or effective length of the releasing device 37, in particular in axial direction and/or along the axis A, may be reduced. This allows in particular that the plunger 4 is first used to move the releasing device 37 relative to the holding device 33, so that the implant 2 is released, and that, subsequently, the implant 2 is pushed out of the needle 5 by the pusher 7.

The spring section 37C preferably comprises or is formed by a preferably at least essentially hollow-cylindrical skeleton having a plurality of openings or breakthroughs 37D. The spring section 37C or skeleton preferably extends along and/or surrounds the axis A.

Due to the plurality of openings or breakthroughs 37D, the spring section 37C or skeleton is preferably compressible.

In particular, when the plunger 4 is moved towards the releasing device 37, the holding device 33 is lifted, in particular from the implant 2, as described before. Then, the releasing device 37 preferably contacts the wall of the interior 3C, so that the releasing device 37 is not further moveable. At this point, when the plunger 4 is further moved towards the releasing device 37, the spring section 37C preferably compresses, thus allowing a further movement of the plunger 4. Then, the pusher 7 will contact the implant 2 and eventually push the implant 2 out of the needle 5.

As an alternative or in addition to the holding device 33, the implant 2 may be held in the needle 5 and/or in position by an in particular sticky fluid, paste or salve. For example, the implant 2 may be held in the needle 5 and/or in position by vaseline/petroleum jelly.

It is preferred that the implanting device 1 be configured in such a way that a force threshold has to be exceeded for moving the plunger 4 out of the initial position and/or towards the needle 5 and/or connecting device 6. In particular, the plunger 4 is latched in the initial position, preferably in order to provide the force threshold.

Thus, the implanting device 1 preferably comprises a latching mechanism 26 for latching the plunger 4 in the initial and/or the end position. Preferably, the latching mechanism 26 is configured for latching the plunger 4 both in the initial position and in the end position.

The latching mechanism 26 preferably has one or more notches 16A and one or more cams 26B, which in particular correspond to or are complementary to the notches 26A. Preferably, the plunger 4 comprises the one or more notches 26A and the housing 3 and/or the actuator 8 comprises the one or more cams 26B or vice versa. The cam/cams 26B preferably project(s) from the wall 3D of the housing 3 and/or into the interior 3C.

Preferably, the latching mechanism 26 comprises a notch 26A and a corresponding or complementary cam 26B configured for latching the plunger 3 in the initial position. Alternatively or additionally, the latching mechanism 26 preferably comprises a notch 26A and a corresponding or complementary cam 26B configured for latching the plunger 3 in the end position.

Particularly preferably, the latching mechanism 26 comprises two notches 26A and two cams 26B, wherein the notches 26A and the cams 26B form two pairs, each comprising one of the notches 26A and one of the cams 26B. One of the pairs is preferably configured for latching the plunger 4 in the initial position and the other pair is preferably configured for latching the plunger 4 in the end position.

The force threshold to be exceeded for moving the plunger 4 out of the initial position is in particular caused by the latching mechanism 26 and/or the latching of the cams 26B at the plunger 4 in the initial position.

The actuator 8, in particular the connecting section 13, preferably has one or more projections 13A. The projections 13A are preferably configured for, in particular interlockingly, fixing the actuator 8, in particular the connecting section 13, to the housing 3. The projections 13A preferably reach through the wall 3D and/or are arrangeable or arranged in breakthroughs 3E of the housing 3 configured for receiving the projections 13A and/or fixing the actuator 8, in particular the connecting section 13, to the housing 3. The protections 13A are particularly designed as bolts extending from the connecting sections 13.

In the embodiment shown in particular in FIGS. 4 to 6, each of the one or more cams 26B is preferably formed by a projection 13A of the actuator 8, in particular the connecting section 13.

Thus, the projections 13A preferably have a double function of, on the one hand, fixing the actuator 8 to the housing 3, and, on the other hand, forming a part, in particular the cams 26B, of the latching mechanism 26 for latching the plunger 4 in the initial position. In this way, a simple construction of the implanting device 1 is achieved and the number of parts is reduced.

However, it is not mandatory that the actuator 8 comprises or forms the cams 26B and/or that the projections 13A form cams 26B of the latching mechanism 26 comprises or forms of the actuator 8. For example, the projections 13A may also be projections 13A of the wall 3D itself.

The implanting device 1 preferably comprises an air passage 27. The air passage 27 is in particular arranged between the plunger 4 and the housing 3, in particular the inner wall 3D. The air passage 27 is preferably configured to allow the passage of air between the plunger 4 and the housing 3, in particular the inner wall 3D, and/or to allow air to exit from the implanting device 1 or housing 3. The air passage preferably extends along and/or at least parallel to the axis A. The air passage 27 is particularly configured to avoid an increasing pressure in the implanting device 1, in particular inside the housing 3 and/or in the interior 3C, when moving the plunger 4 towards the axial end 3A of the housing and/or the needle 5 and/or the connecting device 6.

The air passage 27 is in particular formed by a distance and/or free space between the plunger 4 and the inner wall 3D of the housing 3, in particular surrounding the plunger 4 (radially). Thus, the air passage 27 is preferably at least essentially annular.

The implanting device 1, in particular the plunger 4 and/or the housing 3, is/are arranged and/or configured in such a way that the movement of the plunger 4 relative to the housing 3 is impeded by friction. This may be achieved in various ways.

For example, in the first embodiment, the projections 13A of the actuator 8 or connecting section 13 may be configured to contact the plunger 4 during its movement from the initial position to the end position, thus causing a friction. However, other solutions are also possible here. For example, the plunger 4 and/or the housing 3, in particular the inner wall 3D, might comprise one or more projections as an alternative or in addition to the projections 13A of the actuator 8 or connecting section 13.

The implanting device 1 preferably comprises a blocking device 38 for preventing or blocking an, in particular accidental, actuation of the implanting device 1 and/or actuator 8, in particular a pressing down of the actuator 8 and/or the first arm 10.

The blocking device 38 is preferably configured for preventing a movement of the pusher 7 and/or plunger 4 towards the needle 5 and/or implant 2, in particular in addition to preventing an actuation of the actuator 8.

Preferably, the blocking device 38 is arrangeable or arranged at least partly inside the implanting device 1 and/or housing 3 and/or interior 3C and/or between the plunger 4 and the connecting device 5B, in particular in axial direction. Further, the blocking device 38 is preferably removable from the implanting device 1 and/or housing 3 and/or interior 3C. The implanting device 1 and/or housing 3 preferably comprises an opening in which the blocking device 28 is at least partly arranged or arrangeable.

The blocking device 38 is preferably arranged or arrangeable between the actuator 8, in particular the first arm 10, and the housing 3 and/or the connecting device 5B, in particular so that an actuation or pressing down of the actuator 8 and/or first arm 10 is prevented or blocked by the blocking device 38.

The blocking device 38 is preferably connectable to the connecting device 5B, for example by an interlocking connection and/or a force fit. This facilitates assembly of the implanting device, as will be described in further detail below.

The connection between the blocking device 38 and the connecting device 5B is preferably releasable, in particular without destroying or damaging the blocking device 38 and the connecting device 5B.

It is preferred that the blocking device 38 has a recess 39. The recess 39 is preferably configured for receiving and/or encompassing the pusher 7 and/or the needle 5.

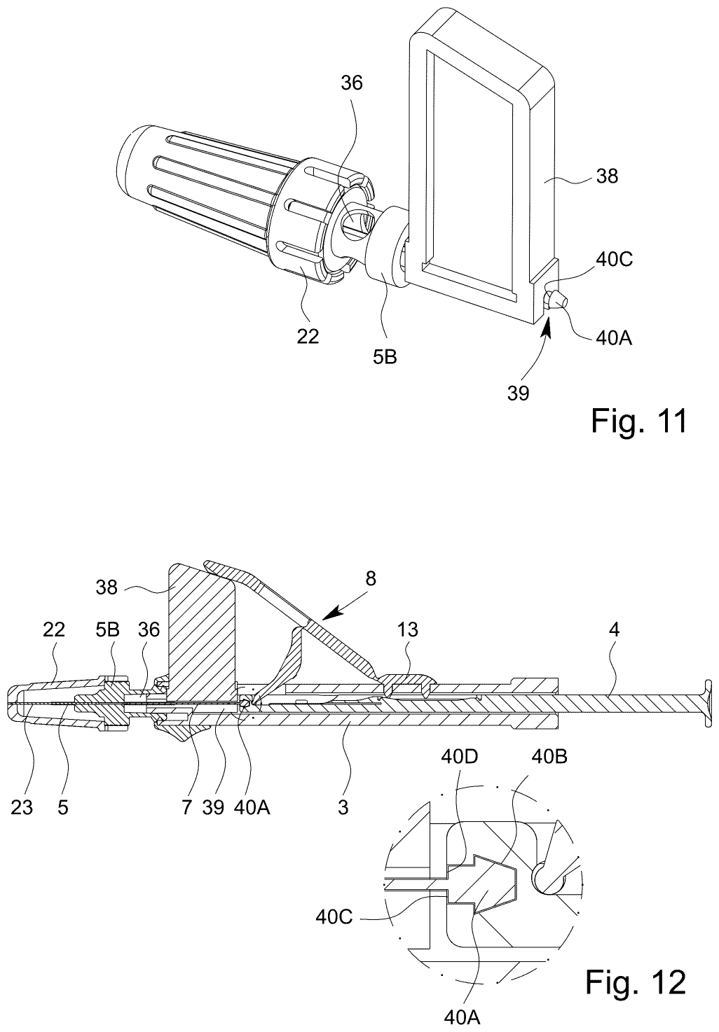

In the embodiment shown in FIGS. 11 and 12, in which the implanting device 1 comprises the blocking device 38, the pusher 7 and the plunger 4 are preferably separate components. In particular, the pusher 7 and the plunger 4 are connectable to each other by interlocking. For this, the pusher 7 and the plunger 4 preferably have interlocking sections 40A, 40B, which correspond to each other.

Preferably, the pusher 7 comprises or forms the interlocking section 40A and the plunger 4 comprises or forms the interlocking section 40B. The interlocking sections preferably form a male part and a female part of an interlocking connection. Particularly preferably, the interlocking section 40A forms a male part and the interlocking section 40B forms a female part of an interlocking connection. However, other solutions are also possible here.

In the embodiment shown, the interlocking section 40A of the pusher 7 is formed by an at least essentially conical part. The conical part preferably widens towards the side or end of the pusher 7 that faces the implant 2 and/or is received or adapted to be received in the needle 5. Preferably, the interlocking section 40A has an undercut 40C, which is preferably transverse, in particular perpendicular, to the axis A and/or configured for interlocking with the interlocking section 40B of the plunger 4.

The interlocking section 40A and/or conical part and/or undercut 40C preferably has a larger cross section than the recess 39, in particular so that interlocking section 40A cannot be placed within the recess 39 and/or, when the pusher 7 is received within the recess 39, a movement of the pusher 7 towards the recess 39 is blocked by the interlocking section 40A and/or conical part and/or undercut 40C contacting or being blocked by the blocking device 38 and/or not fitting into the recess 39.

The interlocking section 40B is preferably configured to receive the interlocking section 40A and/or shaped complementary to the interlocking section 40A. In particular, the interlocking section 40B is of conical shape and preferably widens towards the pusher 7 and in particular the complementary conical shape. Preferably, the interlocking section 40B has an undercut 40D, which is preferably transverse, in particular perpendicular, to the axis A and/or configured for interlocking with the interlocking section 40A, in particular the undercut 40C, of the pusher 7.

The interlocking sections 40A, 40B are preferably configured for an unreleasable or irreversible connection and/or cannot be separated from one another after they have been connected.

One problem in the assembly of the implanting device 1 is to connect all the parts without damaging the implant 2 and/or other parts and without accidentally moving the implant 2 inside the needle 5 or even pushing the implant 2 out of the needle 5. The combination of the implanting device 1 having the blocking device 38 and the configuration of the pusher 7 and the plunger 4 as two separate parts which can be connected to each other in particular facilitates the assembly of the implanting device 1.

The assembly of the implanting device 1 with the blocking device 28 is preferably as follows:

The implant 2 is placed in the needle 5. The cap 22 with the implant lock 23 may be connected to the needle 5 and/or connecting device 5B before or after placing the implant 2 in the needle 5.

Then, the pusher 7 is preferably inserted into the needle 5. Before or after inserting the pusher 7 into the needle 5, the blocking device 38 is connected to the connecting device 5B and/or placed over the needle 5 and/or the pusher 7 and/or between the pusher 7, in particular the interlocking section 40A, and the connecting device 5B, particularly so that the pusher 7 and/or needle 5 is/are received or arranged in the recess 39.

After that, the other parts of the implanting device 1, in particular the housing 3, plunger 4 and actuator 8, are preferably connected to the assembly with the connecting device 5B, pusher 7 and blocking device 38. In particular, the connection is made by connecting the plunger 4 to the pusher 7 by pushing the plunger 4 towards the pusher 7 so that the interlocking sections 40A, 40B are connected to each other, in particular by snapping in or the like. Further, when the pusher 7 and the plunger 4 are connected to one another, the housing 3 and the connecting device 5B are preferably connected to one another at the same time.

During assembly, the blocking 38 preferably blocks or prevents a movement of the pusher 7 towards the implant 2 by the pusher 7 being received in the recess 39. In particular, a movement of the pusher 7 is prevented by the interlocking section 40A and/or conical part and/or undercut 40C. Thus, preferably, the blocking 38 protects the implant 2 from being contacted, moved and/or damaged by the pusher 7 during assembly. This is in particular advantageous during the step of connecting the plunger 4 to the pusher 7.

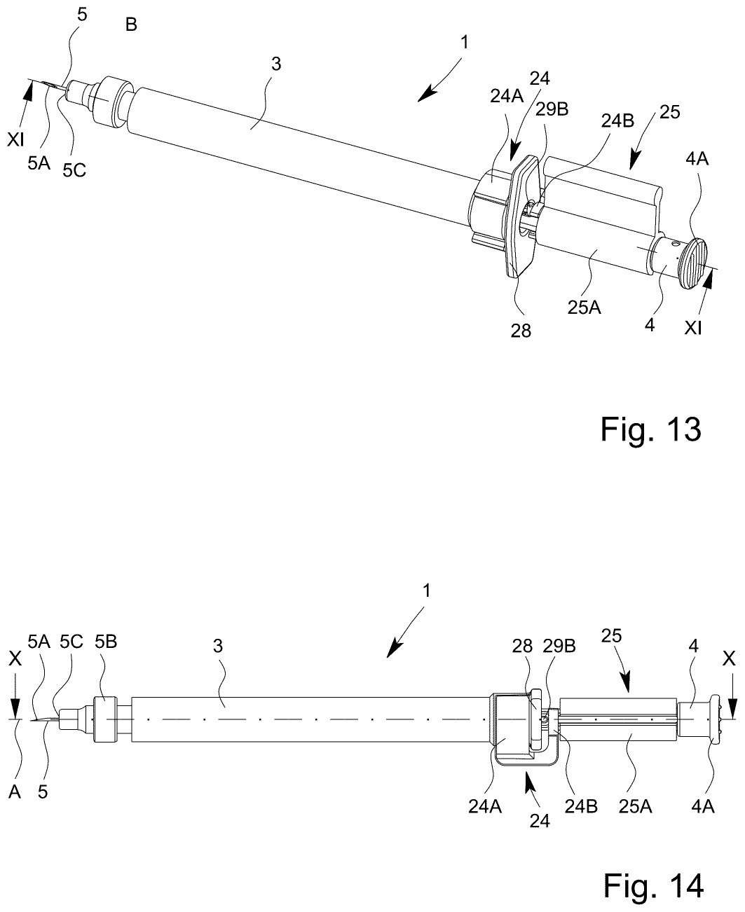

In the following, a second embodiment of the implanting device 1 is described with particular reference to FIGS. 13 to 16.

FIGS. 13 to 16 show a second embodiment of an implanting device 1 according to the present invention. The main difference between the implanting device 1 according to the first embodiment shown in FIGS. 1 to 6 and the second embodiment shown in FIGS. 13 to 16 is in particular that the implanting device 1 according to the second embodiment does not comprise an actuator 8. Apart from that, the implanting device 1 according to the second embodiment preferably has the same or at least most of the features of the implanting device 1 according to the first embodiment and vice versa. Thus, if not explicitly denoted otherwise or apparent from the context, features explained in connection with the first embodiment are also features of the second embodiment and vice versa, even if this is not explicitly mentioned.

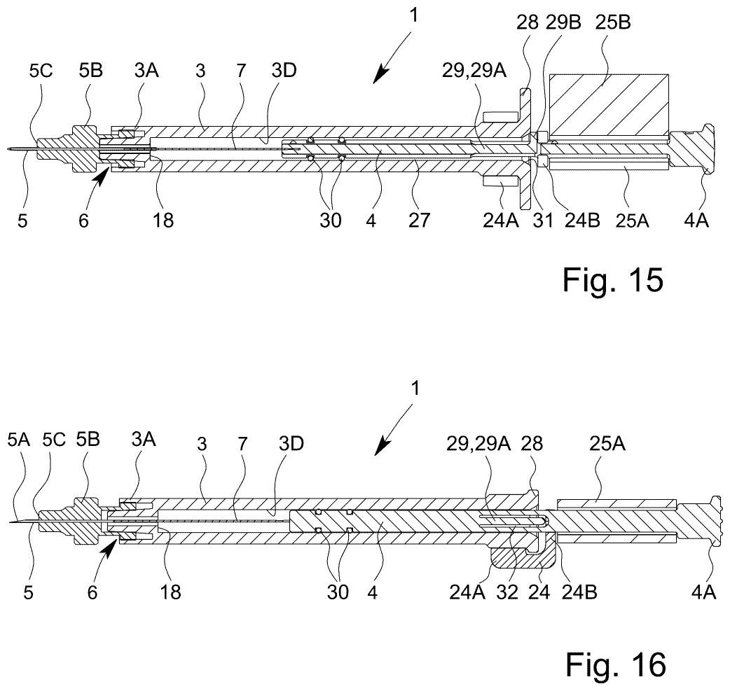

FIG. 13 shows the implanting device 1 according to the second invention in a perspective view. FIG. 14 shows the implanting device 1 according to the second embodiment in a side view. FIGS. 15 and 16 show different cross sections of the implanting device 1 according to the second embodiment.

In the second embodiment, the plunger 4 is preferably movable by pushing against an axial end 4A of the plunger 4 which is in particular arranged opposite an axial end of the plunger 4 facing or directed towards the needle 5 and/or connecting device 6.

The axial end 4A preferably projects laterally from the plunger 4 and/or has a larger diameter and/or cross section perpendicular to the axis A than the other sections of the plunger 4. Preferably, the axial end 4A comprises and/or forms an actuating section of the plunger 4A and/or is configured for actuating and/or moving the plunger 4.

Thus, in the second embodiment, the implanting device 1 is preferably actuatable or actuated and/or movable or moved by pushing against the axial end 4A or actuating section of the plunger 4.

The implanting device 1 preferably has at least one securing device 24, 25. Particularly preferably, the implanting device 1 has at least or exactly two securing devices 24, 25. The securing devices 24, 25 are in the following denoted as “first securing device 24” and “second securing device 25”.

The denotations “first securing device 24” and “second securing device 25” do not imply a sequence or order of the securing devices 24, 25. Further, the term “first securing device” does not necessarily imply the presence of a second securing device. Likewise, the term “second securing device” does not necessarily imply the presence of two securing devices or of a first securing device. Thus, the implanting device 1 may comprise only one of the first and second securing devices 24, 25.

Although described here in connection with the second embodiment, the implanting device 1 according to the first embodiment may also comprise the first securing device 24 and/or second securing device 25.

The first securing device 24 is preferably configured for preventing or blocking an axial movement of the plunger 4 away from the needle 5 and/or the connecting device 6 (i.e. to the right in FIGS. 15 and 16).

The first securing device 24 is preferably designed as a clip or clamp or the like. Preferably, the first securing device 24 is connected or connectable to the housing 3 and/or the plunger 4. Further, the first securing device 24 is preferably removable from the housing 3 and/or implanting device 1, in particular without destroying or damaging the first securing device 24.

It is preferred that the first securing device 24 be made of a flexible and/or elastic material, in particular plastics.

The first securing device 24 is in particular designed to be removable from the implanting device 1, housing 3 and/or plunger 4 by pulling it radially away from the longitudinal axis A. In particular, the first securing device 24 has such a flexibility that, when the first securing device 24 is pulled radially away from the longitudinal axis A, the first securing device 24, in particular the first and/or second gripping second(s) 24A, 24B are deformed and can thus be easily removed from the housing 3, implanting device 1 and/or plunger 4.

The first securing device 24 preferably at least partially surrounds the housing 3 and/or the plunger 4 or is configured therefor.

The first securing device 24 preferably has a first gripping section 24A. The first securing device 24 and/or first gripping section 24A is preferably designed for gripping and/or at least partially encompassing the housing 3.

Further, the first securing device 24 preferably has a second gripping section 24B. The first securing device 24 and/or second gripping section 24B is preferably designed for gripping and/or at least partially encompassing the plunger 4.

The first and/or second gripping sections 24A, 24B preferably has/have a C shape.

The implanting device 1 and/or housing 3 preferably comprises a flange 28, which is in particular arranged at an axial end of the housing 3 opposite the axial end 3A. The flange 28 preferably projects laterally from the housing 3 and/or is arranged transversely, in particular perpendicularly, to the axis A.

The flange 28 preferably forms a stop for the first securing device 24, in particular the first gripping section 24A. In particular, the flange 28 is designed for blocking or preventing a movement of the first securing device 24, in particular the first gripping section 24A, away from the needle 5 and/or connecting device 6.

The plunger 4 preferably comprises a stopping device 29. The stopping device 29 is in particular configured for preventing a movement of the plunger 4 away from the needle 5 and/or connecting device 6. In particular, the stopping device 29 cooperates or is designed to cooperate with the first securing device 24, in particular the second gripping section 24B.

The stopping device 29 is preferably designed as a flexible arm. In particular, the stopping device 29 or arm comprises a longitudinal section 29A, which in particular extends along the axis A, and a transversal section 29B, which in particular extends transversally, in particular perpendicularly, to the axis A and/or the longitudinal section 29A. In particular, the transversal section 29B projects laterally from the plunger 4.

The stopping device 29 or transversal section 29B is in particular configured for resting against the first gripping device 24, in particular the second gripping section 24B.

In particular, the first securing device 24, the flange 28 and the stopping device 29 cooperate in order to prevent a movement of the plunger 4 away from the needle 5 and/or connecting device 6. In particular, a movement of the plunger 4 away from the needle 5 and/or connecting device 6 is prevented by the stopping device 29, in particular the lateral section 29B thereof, against the first securing device 24, in particular the second gripping section 24B thereof, wherein the gripping device 24 is at the same time held in position by the first gripping section 24A resting against the flange 28.

In other words, when the securing device 24 is arranged at and/or connected to the housing 3, the plunger 4 cannot be moved away from the needle 5 and/or connecting device 6 because, upon such an (attempted) movement, the lateral section 29B rests against the second gripping section 24B and the second gripping section 24B does not move because the first securing device 24 is held in position by the first gripping section 24A resting against the flange 28.

The stopping device 29 is preferably flexible, in particular so that a movement of the plunger 4 towards the needle 5 and/or connecting device 6 is allowed and/or is not prevented or blocked by the stopping device 29.

Particularly, when the plunger 4 is moved from the initial position towards the needle 5 and/or connecting device 6, the stopping device 29, in particular the lateral section 29B, comes into contact with the housing 3. When the plunger 4 is then further moved towards the needle 5 and/or connecting device 6, the stopping device 29 preferably flexes or bands or moves inward, so that the contact between the lateral section 29B and the housing 3 does not prevent or block a further movement of the plunger 4.

In particular, the housing 3 comprises a slant 31. The slant 31 and/or the stopping device 29, in particular the transversal section 29B, are configured so that the contact of the stopping device 29 or transversal section 29B leads to an inward movement of the stopping device 29 and/or a sliding movement of the stopping device 29 and/or transversal section 29B along the slant 31.

The stopping device 29, in particular the longitudinal section 29A, is preferably formed and/or allowed to move by a cut 32 in the plunger 4 that forms or defines the stopping device 29 or longitudinal section 29A.

The second securing device 25 is preferably configured for preventing or blocking an axial movement of the plunger 4 towards the needle 5 and/or the connecting device (i.e. to the left in FIGS. 15 and 16).

The second securing device 25 is preferably arranged or arrangeable between the axial end 4A of the plunger 4 and the housing 3, in particular the flange 28, and/or the first securing device 24, in particular the second gripping section 24B.

The second securing device 25 is preferably designed as a clip or clamp or the like. Preferably, the second securing device 25 is connected or connectable to the plunger 4. Further, the second securing device 25 is preferably removable from the plunger 4 and/or implanting device 1, in particular without destroying or damaging the second securing device 25.

It is preferred that the second securing device 25 be made of a flexible and/or elastic material, in particular plastics.

The second securing device 25 preferably at least partially surrounds the plunger 4 or is configured therefor.

The second securing device 25 preferably has a gripping section 25A. The second securing device 25 and/or gripping section 25A is/are preferably designed for gripping and/or at least partially encompassing the plunger 4.

The gripping section 25A preferably has a C shape.

The second securing device 25 preferably has picking section 25B. The picking section 25B is preferably configured for grabbing or picking by a user, in particular in order to remove the second securing device 25 from the implanting device 1 or plunger 4, particularly by pulling the second securing device 25 radially away from the implanting device 1 or plunger 4.

The picking section 25B is preferably designed as a sheet or plate and is in particular flat. Preferably, the picking section 25B is thickened at an end opposite the gripping section 25A.

In the second embodiment, the afore-mentioned friction between the plunger 4 and the housing 3 or inner wall 3D is preferably produced by one or more, in particular two, O-rings 30. The O-rings 30 preferably surround the plunger 4 and are in particular in contact with the housing 3 or inner wall 3D.

The O-rings 30 may also be present in the first embodiment, although not shown in the figures. Further, the second embodiment may also have, in addition or as an alternative to the O-rings 30, have the same devices for producing in the friction between the plunger 4 and the housing 3 as described in connection with the first embodiment.

The O-rings 30 preferably have one or more grooves (not shown). The grooves preferably allow air to pass between the O-rings 30 and the housing 3 or wall 3D and/or form the air passage 27 or a part thereof.

List of reference numbers

| | |
|---|---|
| 1 | implanting device |
| 2 | implant |
| 3 | housing |
| 3A | axial end of 3 |
| 3B | opening of 3 |
| 3C | interior of 3 |
| 3D | (inner) wall of 3 |
| 3E | breakthrough of 3 |
| 4 | plunger |
| 4A | axial end of 4 |
| 5 | (hollow) needle |
| 5A | bevel |
| 5B | connecting device of 5 |
| 5C | contact area |
| 5D | opening of 5 |
| 6 | connecting device of 1 |
| 7 | pusher |
| 8 | actuator |
| 9 | lever mechanism |
| 10 | (first) arm |
| 10A | fixed end of 10 |
| 10B | free end of 10 |
| 10C | first section of 10 |
| 10D | second section of 10 |
| 11 | (second) arm11A first end of |
| 11 | |
| 11B | second end of 11 |
| 12 | solid joint |
| 13 | connecting section |
| 13A | projection |
| 14 | solid joint |
| 15 | toggle joint |
| 15A | first bar of 15 |
| 15B | second bar of 15 |
| 15C | joint of 15 |
| 15D | fulcrum of 15 |
| 16 | second class lever |
| 16A | fulcrum of 16 |
| 17 | stop |
| 18 | stop |
| 19 | grip |
| 20 | coupling device (actuator) |
| 21 | coupling device (plunger) |
| 22 | cap |
| 23 | implant lock |
| 24 | (first) securing device |
| 24A | (first) gripping section of 24 |
| 24B | (second) gripping section of |
| 25 | (second) securing device |
| 25A | gripping section of 25 |
| 25B | picking section of 25 |
| 26 | latching mechanism |
| 26A | notch of 26 |
| 26B | cam of 26 |
| 27 | air passage |
| 28 | flange |
| 29 | stopping device |
| 29A | longitudinal section of 29 |
| 29B | transversal section of 29 |
| 30 | O-ring |
| 31 | slant |
| 32 | cut |
| 33 | holding device |
| 33A | holding section |
| 33B | connecting section |
| 33C | contact section |
| 34 | holder |
| 35 | indicator |
| 36 | opening |
| 37 | releasing device |
| 37A | contact area |
| 37B | recess |
| 37C | spring section |
| 37D | ring-like elements |
| 38 | blocking device |
| 39 | recess |
| 40A | interlocking section |
| 40B | interlocking section |
| 40C | undercut |
| 40D | undercut |
| A | axis |

1-15. (canceled)

16. Implanting device for inserting an implant into tissue, in particular an eyeball, the implanting device having a housing, a plunger received at least partly in the housing and, at an axial end of the housing, a hollow needle and/or a connecting device for connecting a hollow needle to the housing, the plunger being axially movable relative to the housing and/or the needle,
characterized in that
the implanting device has an actuator for moving the plunger relative to the housing, the actuator having or forming a lever mechanism, and/or
the needle is freely rotatable around its longitudinal axis and relative to the housing, and/or the implanting device has a holding device for holding an implant received in the needle in position, and/or
the implanting device has a cap configured to cover the needle, the cap comprising an implant lock for keeping the implant inside the needle.

17. Implanting device according to claim 16, characterized in that the actuator has or forms a toggle joint.

18. Implanting device according to claim 16, characterized in that the actuator has an arm forming a second class lever.

19. Implanting device according to claim 16, characterized in that the actuator has an arm with a first section and a second section, the first section forming a bar of a toggle joint and the second section forming an actuating section.

20. Implanting device according to claim 16, characterized in that the actuator has an arm which is configured and/or arranged to directly act on the plunger so as to move the plunger when the actuator is actuated, in particular in an axial direction and/or towards the needle and/or connecting device.

21. Implanting device according to claim 16, characterized in that the actuator is a one-piece component and/or has two arms which are in particular formed in one piece and/or are connected by a solid joint, in particular a film hinge.

22. Implanting device according to claim 16, characterized in that the actuator is configured to snap in with the plunger when the plunger reaches or has reached an end position, preferably wherein the snapping in produces a tactile, visible and/or audible signal.

23. Implanting device according to claim 16, characterized in that the plunger is latched in an initial position and/or the implanting device is configured in such a way that a force threshold has to be exceeded for moving the plunger out of the initial position and/or towards the needle and/or connecting device.

24. Implanting device according to claim 16, characterized in that the implanting device comprises a grip that is rotatably connected or connectable to the housing and connected or connectable to the needle in a rotationally fixed manner, in particular so that the needle may be freely rotated relative to the housing by a rotation of the grip.

25. Implanting device according to claim 16, characterized in that the plunger and/or the housing are arranged and/or configured in such a way that a movement of the plunger relative to the housing is impeded by friction.

26. Implanting device according to claim 16, characterized in that the implanting device comprises an air passage between the plunger and the housing.

27. Implanting device according to claim 16, characterized in that the implanting device has, in particular at an end opposite the needle and/or the connecting device, at least one securing device, in particular a first securing device for preventing or blocking an axial movement of the plunger towards the needle and/or the connecting device and/or a second securing device for preventing or blocking an axial movement of the plunger away from the needle and/or the connecting device.

28. Implanting device according to claim 16, characterized in that the implanting device has a blocking device for blocking an actuation of the implanting device and/or actuator, in particular a pressing down of the actuator and/or the first arm thereof, preferably wherein the blocking device is additionally configured for preventing a movement of plunger towards the needle and/or implant.

29. Implanting device according to claim 16, characterized in that the housing is at least partly translucent or transparent, in particular so that the plunger or a part thereof is visible through the housing, and/or in that the implanting device has an indicator for indicating the status and/or position of the holding device and/or implant, the indicator preferably being visible through an opening of the implanting device and/or housing and/or connecting device.

30. Implanting device according to claim 16, characterized in that the holding device is designed as an elastic element, in particular a spring.
